(19) United States
(12) Patent Application Publication
(10) Pub. No.: US 2017/0245570 A1
(43) Pub. Date: Aug. 31, 2017

(54) SMART GARMENT

(71) Applicants: William Yuen, Miami Beach, FL (US); Jack Robert Yuen, Darien, CT (US)

(72) Inventors: William Yuen, Miami Beach, FL (US); Jack Robert Yuen, Darien, CT (US)

(21) Appl. No.: 15/440,783

(22) Filed: Feb. 23, 2017

Related U.S. Application Data
(60) Provisional application No. 62/299,832, filed on Feb. 25, 2016.

(51) Int. Cl. A41H 1/02; A41D 1/00; A41B 1/08; A41B 9/00; A41B 11/00; A41C 5/00; A41D 1/06; A41D 1/14; A41D 1/22; A41D 10/00; A41D 13/00; A41D 13/02; A41D 1/18; A41D 19/04; A41D 19/015; A42C 1/00; A42C 2/00; A42C 3/06; A43D 1/02; A43B 7/32; A41D 13/05; A41D 13/06; A61B 5/00; A61B 5/01; A61B 5/021; A61B 5/1477; G06T 19/20; F41H 1/02; F41H 1/04; A41H 3/00

(52) U.S. Cl. CPC A41H 1/02; A41H 3/007; A41D 1/002; A41B 1/08; A41B 9/00; A41B 11/00; A41C 5/00; A41D 1/06; A41D 1/14; A41D 1/22; A41D 10/00; A41D 13/0015; A41D 13/02; A41D 1/18; A41D 19/046; A41D 19/015; A42C 1/00; A42C 2/007; A42C 3/06; A43D 1/02; A43B 7/32; A41D 13/0512; A41D 13/0518; A41D 13/065; A61B 5/6804; A61B 5/01; A61B 5/021; A61B 5/1477; A61B 5/4266; G06T 19/20; F41H 1/02; F41H 1/04; G06T 2210/16

(57) ABSTRACT

Articles and methods for obtaining measurements for the design and/or manufacture of personalized and/or customized clothing, and/or alteration of existing patterns or clothing, and for measuring and/or monitoring one or more parameters, such as size(s), shape(s) and dimension(s), of one or more aspects of a body of the wearer of the article. The articles include a stretchable material interwoven with sensors and/or conductive fibers. The articles include socks, bodysuits, bras, gloves, hats, and so forth. The personalized and/or custom-fitted clothing includes shirts, suits, pants, shorts, dresses, skirts, bras and other undergarments, sleepwear, socks, shoes, gloves, hats, exercise/active wear, protective wear (e.g., helmet, shoulder pads, body armor, etc.), and so forth. The articles and methods may further be used for health and fitness assessment.

### CROSS-REFERENCE TO RELATED APPLICATIONS

The present application claims the priority benefit of U.S. provisional patent application No. 62/299,832 filed Feb. 25, 2016, the disclosure of which is incorporated by reference herein.

### BACKGROUND OF THE INVENTION

Field of Invention

The present invention generally relates to garments and clothing. The present invention more specifically related to methods for measuring or monitoring one or more aspects of a body such as size, shape, and dimension for the purpose of designing and manufacturing personalized or custom fitted clothing.

Description of the Related Art

Clothing—fibers and textiles worn on the human body—is a feature of nearly all human societies. The wearing of clothing can be a function of environment (e.g., a heavy coat worn to protect a person from the elements), safety (e.g., specialized reflector gear for highway workers), hygiene (e.g., disposable garments for a hospital worker), or authority (e.g., a military uniform reflecting a particular rank). In many instances, however, clothing is a factor of personal style.

In the instance of personal style and day-to-day wear, clothing is typically available to the consuming public “off the rack,” as an alteration of an “off the rack” garment purchase, or having been individually tailored. “Off the rack” garments are ready-made and mass-produced in standard sizes that would typically accommodate most body sizes of the consuming public. While such garments may accommodate a variety of general body dimensions, it would be inaccurate to say that the clothing actually “fits” every purchaser. The specific body dimensions of one consumer of general size will never be exactly the same as another consumer of the same general size. These minor nuances can result in drastic variations as to how well a garment actually fits, how pleased the purchaser is with said purchase, and the value they are willing to pay for that garment or a similar brand name in the future. This issue is especially pronounced when clothing is purchased on-line.

A second option for purchasing clothing is to have an “off the rack” garment altered. Altered garments are nevertheless an “off the rack” purchase. Instead of accepting the variety of fitting nuances described above, the purchaser takes the “off the rack” article to a tailor or other service provider. The tailor physically alters various measurements of the purchased garment to correspond to the unique physique of the actual purchaser. While the resulting garment is closer to the actual fit of the purchaser, alterations significantly increase the cost of the overall purchase, delay the opportunity to wear the garment, and may result in a physical alteration that makes it impossible to further alter the garment (e.g., over shortening a sleeve or pant leg). And notwithstanding the costs and effort described above, the original design of the garment may hinder or prevent a necessary alteration that is otherwise required to make the garment constitute a perfect fit.

The third means for acquiring a garment addresses the desire of a purchaser to have a more perfectly fitting garment that is made to order and addresses the physical nuances of the purchasing wearer: individual tailoring. Historically, when a purchaser desired personalized or custom-fitted clothing, the purchaser would go to a designer, tailor, or seamstress, have measurements taken, return for at least one if not more follow up fittings. The purchaser would then receive clothing that may or may not fit as desired and, as a result, could actually require additional alterations.

Notwithstanding the increased likelihood of receiving a better fitting garment, many people are uncomfortable with being touched or measured by another person. Tailoring also requires additional time and materials to be utilized due to the inefficiencies and human error associated with the process. This approach also incurs additional costs associated not only with the actual tailoring and fitting but the possibility that the garment need be shipped, re-tailored, re-shipped and, often times, simply not worn for as long as what would otherwise be the lifetime of the clothing as tailored goods are often designed or worn for special “one night only” events such as movie premiers or other red carpet affairs.

A more recent phenomenon has been for purchasers to measure themselves and then send the measurements by mail or electronically (e.g., through an electronic mail message or web form) to a designer or manufacturer that then produces a purported more custom-fit article of clothing. The manufacturer then uses the measurements and a purchaser selected set of clothes, designs, or fabric samples to provide the purchase with the requested clothing product.

This approach, while made easier by the sharing of information, data, and designs over communications networks, nevertheless has drawbacks. For example, the process is imprecise at best and leaves room for human error during not only the manufacturing process but the original measurement process. Additionally, the individual taking the measurements is not the same individual designing and manufacturing the clothing. There may be discontinuity between certain nuances or specifics of the measurement process that may be unique to aspects of the design and manufacture process. This approach further fails to eliminate the discomfort that some persons have in being touched and measured by another person unless that person measures themselves, which is all but impossible for certain measurements.

Even presently known three-dimensional scanning equipment, generally purchased by and installed at retail stores and/or malls, may present difficulties as they may be expensive, bulky, and/or require assistance to use. Improper usage may result in inaccurate data, thereby resulting in ill-fitting garments. Such a result would wholly defeat the purpose of scanning, which is to obtain accurate data regarding the body of the individual for the purposes of constructing custom-fit garments specifically tailored to the individual.

Such approaches as described above may be limited to matching measurements. As a result, such an approach fails to account for how the clothing may fit or hang on a body, nor user preferences regarding the same. An individual may prefer a looser fit for casual or active-wear, for example, but a closer fit for formal evening wear. In that regard, such an approach further fails to take into account how the body moves and the range of movement.

Accordingly, there is a need in the art for improved systems and methods to obtain personalized or custom-fitted clothing. Such improvements would reduce or eliminate the human error factor in both the measurement and tailoring/manufacture steps. Such improvements would similarly reduce or eliminate the need for third-party human contact while simultaneously decreasing time to and cost of delivery.

### SUMMARY OF THE PRESENTLY CLAIMED INVENTION

In a first claimed embodiment of the present invention, an apparatus for designing custom-fit clothing and/or alteration of existing patterns or clothing is recited. The apparatus includes a stretchable material fitted on at least a portion of the body. A sensor having a conductive fiber is interwoven into the stretchable material. The sensor receives measurement data from the portion of the body fitted with the stretchable material, the measurement data concerns a measurement of one or more parameters of the portion of the body fitted with the stretchable material. The measurement data is used for design of custom-fit clothing and/or alteration of existing patterns or clothing. The apparatus also includes a data network interface communicatively coupled to the conductive fiber.

A second claimed embodiment of the present invention utilizes the aforementioned apparatus to obtain measurements.

A third claimed embodiment of the present invention utilizes the aforementioned apparatus to manufacture custom-fit clothing and/or alter existing patterns or clothing.

### BRIEF DESCRIPTION OF THE DRAWING

FIG. 1 is a schematic illustrating an exemplary environment in which a smart garment may be used.

FIG. 2 is a flowchart illustrating an exemplary method for a smart garment.

FIG. 3 is a block diagram of an exemplary computing device that may be used to implement an embodiment of the present invention.

### DETAILED DESCRIPTION

The present invention relates to articles for the design and/or manufacture of personalized and/or custom-fitted clothing and/or alteration of existing patterns or clothing. The articles may comprise garments and are capable of obtaining measurements of the wearer, wherein the measurements may be used to design and manufacture personalized and/or custom-fitted clothing and/or alter existing patterns or clothing. The articles may include, but are not limited to, socks, bodysuits, bras, gloves, hats, and so forth. In some instances, the articles may include a compressible pad that is pressed against a body part to be evaluated (e.g. feet standing on a pad). The articles include a stretchable material interwoven with sensors and/or conductive fibers that are capable of determining one or more parameters, including but not limited to, the size(s), shape(s) and dimension(s) of the wearer for the particular area of the wearer's body covered by the article. The personalized and/or custom-fitted clothing includes, but is not limited to, shirts, suits, pants, shorts, dresses, skirts, bras and other undergarments, sleepwear, socks, shoes, gloves, hats, exercise/active wear, protective wear (e.g., helmet, shoulder pads, body armor, etc.), and so forth.

The present invention further relates to methods for obtaining measurements for designing and manufacturing personalized and/or custom-fitted clothing and/or altering existing patterns or clothing. The method includes the use of one or more articles that may comprise garments and may include, but are not limited to, a sock, bodysuit, bra, glove, and so forth. The articles include a stretchable material interwoven with sensors and/or conductive fibers that are capable of determining one or more parameters, including but not limited to, the size(s), shape(s) and dimension(s) of the wearer for the particular area of the wearer's body covered by the article. The personalized and/or custom-fitted clothing includes, but is not limited to, bras, shoes, gloves, protective wear (e.g., helmets, shoulder pads, body armor, etc.), and so forth. The personalized and/or custom-fitted clothing includes, but is not limited to, shirts, suits, pants, shorts, dresses, skirts, bras and other undergarments, sleepwear, socks, shoes, gloves, hats, exercise/active wear, protective wear (e.g., helmet, shoulder pads, body armor, etc.), and so forth.

Measuring and monitoring in the scope of the present invention may also apply to health, safety, and fitness. For example, sensors in a body suit could detect body temperature, blood pressure, pulse, sweat or other chemicals emitted by the body. Coupled to a computing device with requisite analytical software or other hardware as might be required for a particular sensor and/or detection, the combination of sensor, hardware, and software can measure or analyze a particular body emission or other indicia. Based on this information, certain decisions might be made with respect to a diet or health care, including urgent health care.

Another application involving the present invention might include using a bodysuit where the embedded conductive fiber can provide output such as heat, vibration, electrical stimulation, or even deliver medicinal treatment. For example, such a suit and fiber/emitter combination might be used to raise body temperature in a controlled fashion either by temperature and/or with respect to a particular part of the body.

The aforementioned suit might also be used for fitness and training applications whereby sensor data—including motion tracking data—might be used to analyze and/or measure performance, motion, techniques, or other data. This could and most likely would include information not otherwise perceptible to the human eye including tension, torque, weight, balance, speed, pressure, or acceleration.

The present invention further relates to methods for manufacturing articles that may be used for designing and manufacturing personalized and/or custom-fitted clothing and/or alteration of existing patterns or clothing. The manufactured articles are capable of obtaining measurements of the wearer that may be used to design and manufacture personalized and/or custom-fitted clothing. The manufactured articles may comprise garments and may include, but are not limited to, a sock, bodysuit, bra, glove, and so forth. The manufactured articles include a stretchable material interwoven with sensors and/or conductive fibers that are capable of determining one or more parameters, including but not limited to, the size(s), shape(s) and dimension(s) of the wearer for the particular area of the wearer's body covered by the article. The personalized and/or custom-fitted clothing that may be designed and manufactured using the articles according to the present invention includes, but is not limited to, shirts, suits, pants, shorts, dresses, skirts, bras and other undergarments, sleepwear, socks, shoes, gloves, hats, exercise/active wear, protective wear (e.g., helmet, shoulder pads, body armor, etc.), and so forth.

The present invention relates to articles for measuring and/or monitoring an entire body and/or individual body part(s) of the wearer. The articles may comprise garments, which may include, but are not limited to, socks, bodysuits, bras, gloves, hats, and so forth. The articles may comprise a stretchable material interwoven with sensors and/or conductive fibers that are capable of determining one or more parameters, including but not limited to, the size(s), shape(s) and dimension(s) of the particular area of the wearer's body covered by the article.

The present invention further relates to methods for measuring and/or monitoring an entire body and/or individual body parts. The method comprises an article that may further comprise a garment or garment-like material and may include a stretchable material interwoven with sensors and/or conductive fibers that are capable of determining one or more parameters, including but not limited to, the size(s), shape(s) and dimension(s) of the wearer for the particular area of the wearer's body covered by the article.

The present invention further relates to methods for manufacturing articles that may be used for measuring and/or monitoring an entire body and/or individual body parts. The articles may comprise garments, which may include, but are not limited to, socks, bodysuits, bras, gloves, hats, and so forth. The articles may comprise a stretchable material interwoven with sensors and/or conductive fibers that are capable of determining one or more parameters, including but not limited to, the size(s), shape(s) and dimension(s) of the particular area of the wearer's body covered by the article.

The articles and methods described herein are directed towards obtaining measurements of the wearer of the article(s), wherein the measurements may be used to design and manufacture personalized and/or custom-fitted clothing and/or alteration of existing patterns or clothing or, alternatively, to measure and or monitor the body of the wearer or particular aspects thereof for health and/or fitness purposes. The articles may comprise garments or garment-like material and may include, but are not limited to, socks, bodysuits, bras, gloves, hats, and so forth.

The articles may further comprise a stretchable material comprised of any material(s) known and appreciated by one of skill in the art. In one preferred embodiment, the stretchable material is flexible, elastic and reusable. In one aspect of this preferred embodiment, the stretchable material is capable of conforming to a variety of body or body part sizes, depending on the size of the wearer, such that the article is rendered practically one-size-meets all. The conductive fibers and/or sensors may be placed throughout the article or in particular locations of the article as needed and as would be understood and appreciated by one of ordinary skill in the art.

The stretchable material may further be interwoven with sensors and/or conductive fibers that are capable of determining one or more parameters, including but not limited to, the size(s), shape(s) and dimension(s) of the wearer for the particular area of the wearer's body covered by the article. The sensors and/or conductive fibers may be capable of wired and/or wireless communication and may be comprised of any materials known and understood by one of ordinary skill in the art.

The at least one sensor may be from a plurality of sensors, each of the plurality of sensors having at least one conductive fiber interwoven into the stretchable material, each of the plurality of sensors receiving measurement data from the portion of the body fitted with the stretchable material, the measurement data concerning a measurement of one or more parameters of the portion of the body fitted with the stretchable material, the measurement data used for design of custom-fit clothing and/or alteration of existing patterns or clothing. The at least one sensor may also or alternatively be part of a sensor array, the sensor array having at least one conductive fiber interwoven into the stretchable material, the sensor array receiving measurement data from the portion of the body fitted with the stretchable material, the measurement data concerning a measurement of one or more parameters of the portion of the body fitted with the stretchable material, the measurement data used for design of custom-fit clothing and/or alteration of existing patterns or clothing.

The measurements obtained by the conductive fibers and/or sensors is in the form of data, which may be transmitted via a network interface that might be wired, USB, wireless, or other modes of data transmission as would be known and appreciated by one of skill in the art, and may be transmitted to a computer, handheld device, smart device, phone, server, or any other device as may be known and understood by one of skill in the art. The data may then be uploaded to a graphical software program capable of creating a pattern and/or 2D and/or 3D image of the particular area(s) of the body that were measured, together with corresponding raw data indicating the particular parameters measured in numerical form. The 2D and/or 3D image and/or numerical data may then be used individually or together to create a pattern, physical mold or mannequin or digital “virtual body shape” to replicate the customer's body or body part from which the desired personalized and/or customized clothing can be made to exact or near-exact specifications.

The personalized and/or custom-fitted clothing and/or alteration of existing patterns or clothing includes, but is not limited to, shirts, suits, pants, shorts, dresses, skirts, bras and other undergarments, sleepwear, socks, shoes, gloves, hats, exercise/active wear, protective wear (e.g., helmet, shoulder pads, body armor, shoes or boots, gloves, or chest and knee pads), and so forth.

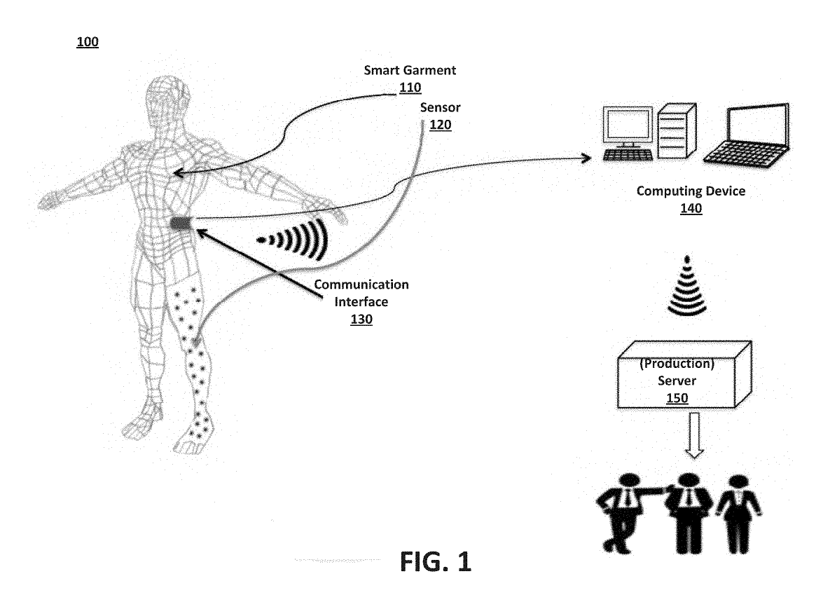

FIG. 1 is a schematic illustrating an exemplary environment 100 in which a smart garment may be used. Such an environment 100 may include a smart garment 110 (which may include one or more sensors 120 and communication interfaces 130), one or more computing devices 140, and a remote server 150.

One embodiment of environment 100 may be directed towards creating tailor-made shoes and clothing without the customer ever having to step into a store. A smart garment 110 may be made of a stretchable material interwoven throughout with sensors 120 (and/or conductive fibers). Such a smart garment 110 may be formed as a bodysuit, stocking/sock, or any other type of garment known in the art.

Smart garment 110 may include one or more sensors 120 and one or more communication interfaces 130, as well as a power source (not pictured). Such a power source may include a battery as known in the art. Alternatively, the smart garment 110 may include a connection that couples to an external power source. Such connections and external power sources may include any that are presently known in the art.

When provided to and worn by the customer, smart garment 110 collects data via sensors 120, which are distributed on different parts of the body. Such data may include various measurements, which may pertain to the physical dimensions of various body parts and their respective movement through three-dimensional space.

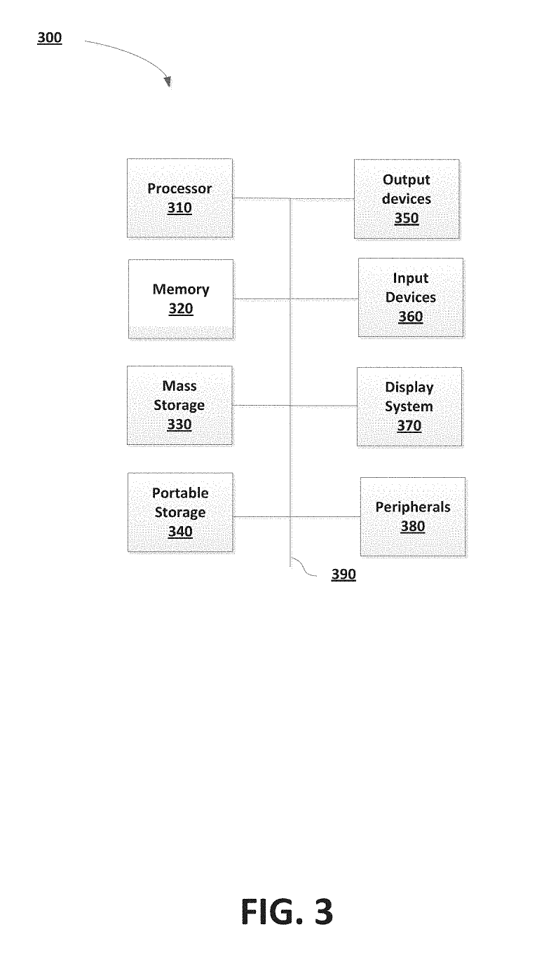

Such data may then be transmitted via a built-in communication device 130 (such as a USB adaptor or wireless capability) to a computing device 140. Computing device 140 may include any type of mobile, handheld, tablet, or desktop computing device known in the art. Such computing devices are described in further detail below with respect to FIG. 3.

Computing device 140 may further include graphic modeling software executable to create a pattern and/or 2D and/or 3D image profile of the body parts wearing the smart garment 110 based on data received from communication interface 130. In this example, the data collected and image created may then be sent to the server 140 (e.g., at a manufacturer or tailor). Such a server 140 may use the 2D or 3D image profile to create a, pattern, mold and/or mannequin and for the design and/or manufacture of personalized and/or customized clothing and/or alteration of existing patterns or clothing, which is then sent to the customer. In some examples, the manufacturer and/or tailor may not require a mold or mannequin to be made but, rather, may use the data measurements and/or the 2D and/or 3D image to design and manufacture the personalized and/or customized clothing and/or alteration of existing patterns or clothing.

The present invention further includes methods for obtaining measurements for the design and manufacture personalized and/or custom-fitted clothing and/or alteration of existing patterns or clothing, further comprising the smart garment 110 as detailed above. In one embodiment of this method, the smart garment 110 is sent to the customer to wear. The sensors 120 on the smart garment 110 may take measurements and transmit the measurements as described above to the server 140 (e.g., associated with a tailor and/or manufacturer of his or her choice). The 2D and/or 3D profile and/or measurement data may then be used to create the customer's body size, shape, or movements and for the design and manufacture of personalized and/or customized clothing and/or alteration of existing patterns or clothing.

In another embodiment, the customer can send new [and old/existing] clothing that has already been purchased [created/manufactured] elsewhere to the tailor and/or manufacturer, together with the data obtained according to article and method of the present invention, for customized and/or personalized modification of the already-purchased clothing. This embodiment may be particularly useful in situations where newly purchased [previously] clothing does not fit properly or as desired or previously purchased clothing no longer fits properly or as desired because of changes in the customer's body parameters (e.g., weight gain, weight loss, weight redistribution, change in body shape, change in height), the customer's desired style or fashion trends.

In another embodiment, the measurement data and/or 2D and/or 3D image profile may be used with a virtual [mirror or] dressing room application. The 2D and/or 3D image profile may be combined with [the individual's facial image, skin tone, hairstyle and] images of differently sized [styled, colored and patterned] clothing to generate composite images of how such clothing may appear when worn by the customer. As such, the customer may evaluate different sizes for fit with respect to various body parts (e.g., shoulders, waist, hips). Because the 2D and/or 3D image profile is based on [the individual's facial image, skin tone, hairstyle and] actual measurements of different body parts (including during movement), the generated composite images represent a more accurate depiction of how such clothing would actually appear if worn by the customer vis-à-vis stock photographs or standard sizing models.

The present invention further relates to articles and methods for measuring and/or monitoring an entire body and/or individual body part(s) of the wearer, wherein the articles and capabilities are as described above. The articles and methods of the present invention may be used for purposed of health and fitness management.

For example, smart garment 110 may comprise golfing gloves whose sensors 120 can track different measurements of the hands during movement (e.g., gripping and swinging), as well as a bodysuit to track movement of the wearer's hips and shoulders during movement. Such data capture by the sensors 120 throughout the course of a movement may be sent by communication interface 130 to computing device 140 to create a 2D and/or 3D image profile that includes movement. Such a 2D and/or 3D image profile may include a 2D and/or 3D model of the wearer of the smart garment 110 throughout the course of a movement (e.g., from a initial stance to follow-through of a swing). Such a 2D and/or 3D image profile may be compared to profiles of other wearers (e.g., professional athletes) for educational or training purposes.

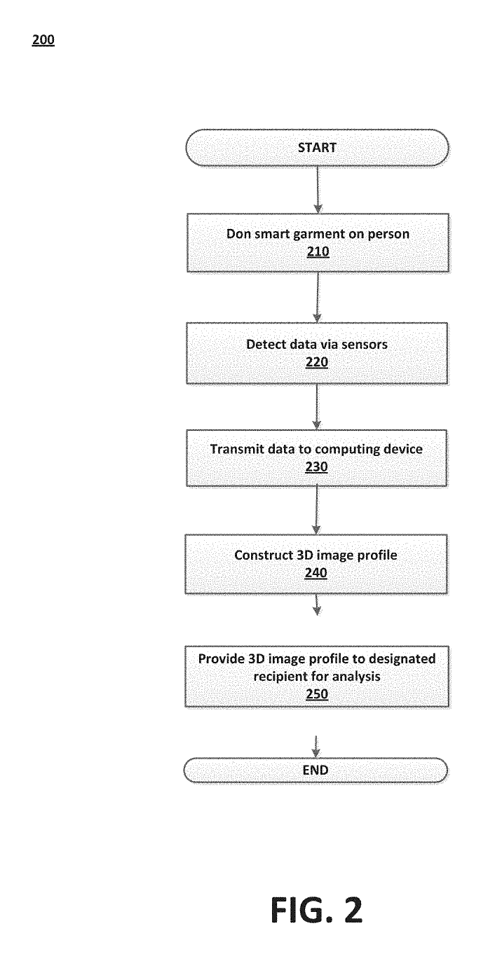

FIG. 2 is a flowchart illustrating an exemplary method 200 for a smart garment. In the method 200, a smart garment may be donned by a user, data may be detected by sensors and sent to a computing device for construction of a 2D and/or 3D image profile, which may then be provided to a designated recipient device for further analysis and processing.

In step 210, a smart garment (e.g., smart garment 110) is donned onto a body of a person. As noted above, such a smart garment 110 may be formed into any type of garment known in the art. Generally, smart garment 110 is made of stretchable material that conforms to the body of the wearer. As such, the sensors (e.g., sensors 120, which are inclusive of conductive fibers) that are distributed throughout the smart garment 110 lie close to the body while the smart garment 110 is worn.

In step 220, the sensors 220 capture data regarding the body of the wearer. Such data may include sensor positions (relative or absolute), sensor movement through space, and various measurements related thereto. Such data may be captured while the body of the wearer is posed in different still positions (e.g., sitting, standing), as well as during movement. Measurements regarding distances and relative orientations between worn sensors, for example, may be captured and compared to corresponding distances and orientations when no one is wearing the smart garment 110.

In step 230, the data captured by the sensors 120 are transmitted by communication interface (e.g., communication interface 130) to a computing device (e.g., computing device 140). Communication interface 130 may include any communication means (wired or wireless) known in the art for transferring data from sensors 120 to computing device 140.

In step 240, computing device 140 constructs a 2D and/or 3D image profile of the wearer's body (at least the part of the body that is wearing the smart garment 110) based on the data captured by sensors 120 and transmitted by communication interface 130. Such a 2D and/or 3D image profile may either be a static body or reflect movement of the body.

In step 250, computing device 140 provides the 2D and/or 3D image profile to one or more designated recipients for analysis and subsequent services based thereon. Such subsequent analysis and services may include clothing manufacture according to the 2D and/or 3d image model, as well as virtual dressing room applications and dance/athletic/fitness training applications.

FIG. 3 is a block diagram of an exemplary computing device 300 (e.g., and computing device 140) that may be used to implement an embodiment of the present invention. The computing system 300 of FIG. 3 includes one or more processors 310, memory 320, mass storage 330, portable storage 340, one or more output devices 350, one or more input devices 360, display system 370, one or more peripherals 380, and bus 390.

Main memory 320 stores, in part, instructions and data for execution by processor 310. Main memory 320 can store the executable code when in operation. The system 300 of FIG. 3 further includes a mass storage device 330, portable storage medium drive(s) 340, output devices 350, user input devices 360, a graphics display 370, and peripheral devices 380.

The components shown in FIG. 3 are depicted as being connected via a single bus 390. However, the components may be connected through one or more data transport means. For example, processor unit 310 and main memory 320 may be connected via a local microprocessor bus, and the mass storage device 330, peripheral device(s) 380, portable storage device 340, and display system 370 may be connected via one or more input/output (I/O) buses.

Mass storage device 330, which may be implemented with a magnetic disk drive or an optical disk drive, is a non-volatile storage device for storing data and instructions for use by processor unit 310. Mass storage device 330 can store the system software for implementing embodiments of the present invention for purposes of loading that software into main memory 320.

Portable storage device 340 operates in conjunction with a portable non-volatile storage medium, such as a floppy disk, compact disk or Digital video disc, to input and output data and code to and from the computer system 300 of FIG. 3. The system software for implementing embodiments of the present invention may be stored on such a portable medium and input to the computer system 300 via the portable storage device 340.

Input devices 360 provide a portion of a user interface. Input devices 360 may include an alpha-numeric keypad, such as a keyboard, for inputting alpha-numeric and other information, or a pointing device, such as a mouse, a trackball, stylus, or cursor direction keys. Additionally, the system 300 as shown in FIG. 3 includes output devices 350. Examples of suitable output devices include speakers, printers, network interfaces, and monitors.

Display system 370 may include a liquid crystal display (LCD) or other suitable display device. Display system 370 receives textual and graphical information, and processes the information for output to the display device.

Peripherals 380 may include any type of computer support device to add additional functionality to the computer system. For example, peripheral device(s) 380 may include a modem or a router.

The components contained in the computer system 300 of FIG. 3 are those typically found in computer systems that may be suitable for use with embodiments of the present invention and are intended to represent a broad category of such computer components that are well known in the art. Thus, the computer system 300 of FIG. 3 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

1. An apparatus for designing custom-fit clothing, the apparatus comprising:
a stretchable material fitted on at least a portion of the body;
at least one sensor coupled to a conductive fiber and interwoven into the stretchable material, wherein the sensor receives measurement data from the portion of the body fitted with the stretchable material, the measurement data concerning a measurement of one or more parameters of the portion of the body fitted with the stretchable material, the measurement data used for design of custom-fit clothing; and
at least one data network interface communicatively coupled to the conductive fiber.

2. The apparatus of claim 1, wherein the data network interface is a wired interface.

3. The apparatus of claim 1, wherein the data network interface is a wireless interface.

4. The apparatus of claim 1, wherein the data network interface is a portable storage interface.

5. The apparatus of claim 4, wherein the portable storage interface is a USB interface.

6. The apparatus of claim 1, wherein the data network interface allows for the measurement data to be provided to a computing device physically separate from the stretchable fiber.

7. The apparatus of claim 6, wherein the stretchable fiber provides the measurement data to a mobile device by way of the network interface.

8. The apparatus of claim 6, wherein the stretchable fiber provides the measurement data to a server by way of the network interface.

9. The apparatus of claim 6, wherein the stretchable fiber provides the measurement data to a tablet device by way of the network interface.

10. The apparatus of claim 1, wherein the stretchable material fits over a portion of the body as a sock.

11. The apparatus of claim 1, wherein the stretchable material fits over a portion of the body as a bodysuit.

12. The apparatus of claim 1, wherein the stretchable material fits over a portion of the body as a bra.

13. The apparatus of claim 1, wherein the stretchable material fits over a portion of the body as a glove.

14. The apparatus of claim 1, wherein the stretchable material fits over a portion of the body as a hat.

15. The apparatus of claim 1, wherein the stretchable material fits over a portion of the body as a shirt or blouse.

16. The apparatus of claim 1, wherein the stretchable material fits over a portion of the body as a pair of pants or slacks.

17. The apparatus of claim 1, wherein the stretchable material fits over a portion of the body as a suit.

18. The apparatus of claim 1, wherein the measurement concerns size.

19. The apparatus of claim 1, wherein the measurement concerns shape.

20. The apparatus of claim 1, wherein the measurement concerns dimensions of an area of the portion of the body.

21. The apparatus of claim 1, wherein the custom-fit clothing is one or more of shirts, suits, pants, shorts, dresses, skirts, bras and other undergarments, sleepwear, socks, shoes, gloves, hats, exercise or active wear, or protective wear.

22. The apparatus of claim 1, wherein the custom-fit clothing is protective wear.

23. The apparatus of claim 22, wherein the protective wear is one or more of a helmet, shoulder pads, body armor, shoes, boots, gloves, or chest and knee pads.

24. The apparatus of claim 1, wherein the at least one sensor is from a plurality of sensors, each of the plurality of sensors having at least one conductive fiber interwoven into the stretchable material, each of the plurality of sensors receiving measurement data from the portion of the body fitted with the stretchable material, the measurement data concerning a measurement of one or more parameters of the portion of the body fitted with the stretchable material, the measurement data used for design of custom-fit clothing.

25. The apparatus of claim 1, wherein the at least one sensor is part of a sensor array, the sensor array having at least one conductive fiber interwoven into the stretchable material, the sensor array receiving measurement data from the portion of the body fitted with the stretchable material, the measurement data concerning a measurement of one or more parameters of the portion of the body fitted with the stretchable material, the measurement data used for design of custom-fit clothing.

26. A method for obtaining measurements, the method comprising:
placing a smart garment on at least a portion of the body, the smart garment comprising a stretchable fabric and at least one sensor coupled to a conductive fiber and interwoven into the stretchable material;
capturing a plurality of measurements via the at least one sensor on the portion of the body fitted with the stretchable material, the measurement data concerning a measurement of one or more parameters of the portion of the body fitted with the stretchable material;
transmitting the captured measurements via at least one data network interface communicatively coupled to the conductive fiber, the captured measurements transmitted to a computing device; and
generating a three-dimensional (3D) image profile of the portion of the body based on the captured measurements.

27. The method of claim 26, further comprising manufacturing an article of clothing and/or alteration of existing patterns or clothing based on the 2D and/or 3D image profile.

28. The method of claim 26, further comprising generating a composite model based on the 2D and/or 3D image profile and one or more images of a pattern and/or an article of clothing.

29. The method of claim 26, wherein the measurements are captured during movement of the portion of the body, and wherein the 2D and/or 3D image profile includes a model representative of the movement.

30. The method of claim 29, further comprising evaluating the 2D and/or 3D image profile to generate metrics regarding the represented movement.

31. A method for obtaining measurements, the method comprising:
capturing two-dimensional and/or three-dimensional data regarding a portion of a body of an individual via at least one sensor, wherein the data is captured responsive to an action by the individual; and
transmitting the captured two-dimensional and/or three-dimensional data over a communication network to a designated recipient device.

32. A method for obtaining measurements, the method comprising:
capturing two-dimensional and/or three-dimensional data regarding a portion of a body of an individual via at least one sensor; and
transmitting the captured two-dimensional and/or three-dimensional data over a communication network to a designated recipient device located at a location remote from the sensor.

33. An apparatus for obtaining measurements, the apparatus comprising:
a compressible material;
at least one sensor integrated into the compressible material, wherein the sensor takes one or more measurements when the compressible material is compressed by a body, the measurements concerning one or more physical dimensions of the body causing the compression; and
at least one data network interface communicatively coupled to the sensor.
